(19) United States
(12) Patent Application Publication
(10) Pub. No.: US 2023/0015204 A1
(43) Pub. Date: Jan. 19, 2023

(54) SPRAY DEVICES FOR ANTIPERSPIRANT OR DEODORANT COMPOSITIONS WITH A COMPRESSED GAS PROPELLANT

(71) Applicant: The Procter & Gamble Company, Cincinnati, OH (US)

(72) Inventors: David Frederick Swaile, Cincinnati, OH (US); Matthew John Martin, California, KY (US)

(21) Appl. No.: 17/847,211

(22) Filed: Jun. 23, 2022

Related U.S. Application Data
(60) Provisional application No. 63/216,652, filed on Jun. 30, 2021.

(51) Int. Cl. A61K 8/26; A61K 8/891; A61K 8/37; A61Q 15/00; A61K 8/04; A45D 34/04

(52) U.S. Cl. CPC A61K 8/26; A61K 8/891; A61K 8/37; A61Q 15/00; A61K 8/046; A45D 34/04; A61K 2800/87; A45D 2200/057

(57) ABSTRACT

An aerosol antiperspirant or deodorant product with a dispenser with a dispenser container filled with nitrogen gas and a reservoir filled with antiperspirant or deodorant composition. The composition is dispensed without substantial clogging in the valve assembly or changes to the mass flow rate and/or average particle size distribution.

### TECHNICAL FIELD

One aspect of the invention relates generally to spray devices containing a deodorant or antiperspirant composition and a propellant. Yet another aspect of the invention relates generally to methods of using antiperspirant spray devices.

### BACKGROUND OF THE INVENTION

Antiperspirant and deodorant products can be packaged in an aerosol container, which is under pressure and includes a release valve that is used to emit the pressurized composition into the air as a fine mist propelled by a gas propellant. Typically, the propellant is a liquified hydrocarbon propellant. An advantage of a hydrocarbon propellant is that inside the container there is enough pressure to turn the gas into a liquid. As the product is dispensed, the product level inside the container drops, and more propellant evaporates into the headspace above the product, maintaining an approximately constant pressure, which in turn yields consistent spray properties, such as spray rate and average particle size distribution.

Even though hydrocarbon propellants provide substantial benefits, some consumers would prefer a product in an aerosol spray device with a non-hydrocarbon propellant, such as compressed gases, which can include, but are not limited to, compressed air, nitrogen, inert gases, and carbon dioxide. Nitrogen or carbon dioxide can be especially desirable because it is non-toxic, non-flammable, relatively low in cost and generally inert.

However, it can be difficult to make a consumer acceptable aerosol antiperspirant and deodorant product that uses a compressed gas propellant because, unlike liquified hydrocarbons, the compressed propellant is always in the vapor state and therefore the pressure in the container is reduced as product is dispensed, making it difficult to dispense the product at a consumer acceptable particle size distribution and spray rate over the life of the container. As the pressure inside the container drops, the average particle size distribution increases, eventually releasing globs of product that take too long to dry and do not provide adequate coverage of the underarm, undermining product performance. Further, as the pressure decreases, the spray rate and distance the product travels also decrease, which can make it difficult for the product to reach the user's underarm. Eventually, the pressure can be so low that no product is released at all, even if there is product left in the can.

Therefore, there is a need for an aerosol spray device that contains an antiperspirant or deodorant product and a compressed gas propellant with consistent spray properties, such as spray rate and average particle size distribution for the life of the container.

### SUMMARY OF THE INVENTION

An aerosol antiperspirant or deodorant product comprising: a dispenser comprising: a dispenser container at least partially filled with a compressed gas selected from nitrogen, carbon dioxide, and combinations thereof; wherein the dispenser is fitted with a valve assembly comprising: a mounting cup, one or more gaskets, a valve seat, a spring, a housing, and a dip tube; an ingredient containing reservoir filled with antiperspirant or deodorant composition operatively connected to the dispenser container and the dip tube via a first dip tube and a second tube such that on actuation of the valve assembly the composition and the nitrogen gas travel along the first tube and the second tube respectively, and mix in the valve assembly before exiting the dispenser container via an actuator spray nozzle to an environment or subject; or the nitrogen gas travels along the second tube into the ingredient containing reservoir carries the composition along the first tube where they mix in the valve assembly before exiting the dispenser container (90) via the actuator spray nozzle (200) to an environment or subject; wherein the antiperspirant or deodorant composition comprises a non-volatile silicone fluid having a concentration from about 30% to about 70% by weight of the antiperspirant composition, a deodorant or antiperspirant active, an organoclay material and at least one liquid activation enhancer having a Hansen Solubility Parameter for Hydrogen Bonding, δₕ, between about 2 and about 6 and a light transmittance value greater than 90%.

### BRIEF DESCRIPTION OF THE DRAWINGS

While the specification concludes with claims, it is believed that the same will be better understood from the following description taken in conjunction with the accompanying drawings wherein like numbers illustrate like elements throughout the views and in which:

FIG. 1 is an exploded view of a prior art male (single) bag on valve assembly;

FIG. 2A is an exploded view of a first embodiment of a valve assembly of the present invention;

FIG. 2B is an exploded view of a first embodiment of a dispenser comprising the valve assembly of FIG. 2A;

FIG. 2C is a side elevation of the assembled dispenser of FIG. 2B;

FIG. 2D is a cross sectional view of the dispenser of FIG. 2B;

FIG. 3 is a cross-sectional view showing an example having only one internal bag for the antiperspirant or deodorant composition.

### DETAILED DESCRIPTION

Some consumers like aerosol antiperspirant or deodorant products that are in an aerosol dispenser. Aerosol products are known to quickly deliver antiperspirant or deodorant that is easy to apply, quick drying, and with less risk of the white build-up occurring on clothes. However, many of these products currently use a hydrocarbon propellant, which delivers excellent spray properties, but is not preferred by some consumers.

It can be difficult to use a non-hydrocarbon propellant, like compressed gas, because the pressure drop during operation can prevent the complete emptying of the product from the container and/or can cause inconsistent spray properties including particle size, spray rate, and the distance the composition can travel.

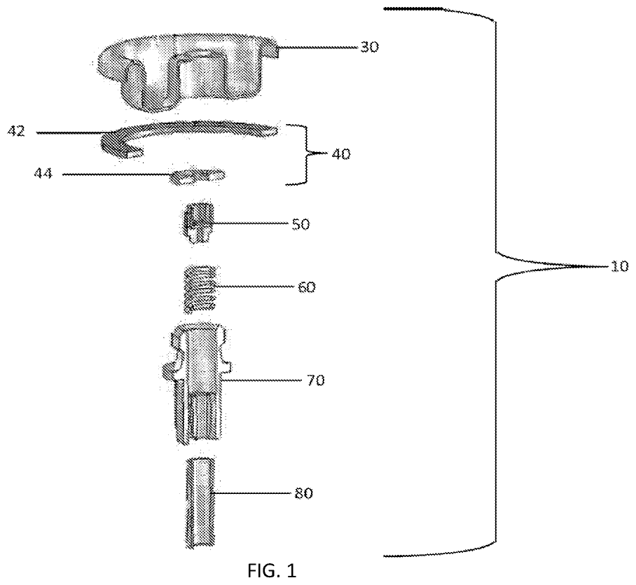

FIG. 1 is a bag on valve assembly (10) comprising a mounting cup (30); an outer (42) and inner (44) gasket (40); a valve seat (50); a spring (60); a housing (70); and a dip tube (80) with a fitment, such as a rib, to which a bag (not shown) is attached.

Various actuators (200) may be connected to the valve assembly (10) which may be a male valve (as illustrated) or a female valve.

In some examples, the aerosol container can have a dispensing system with one ingredient containing reservoir, like a pouch or bag, filled with a product and the spray device container can be filled or partially filled with compressed gas. In this example, a first tube can extend into the containing reservoir and can carry product to the valve assembly and the second tube can extend into the spray device container and can carry compressed gas to the valve assembly. The product and the compressed gas can mix in the valve assembly or actuator. Unlike traditional bag-on-valve executions, when the composition is dispensed, gas is also dispensed from the spray device, decreasing the pressure inside the spray device container.

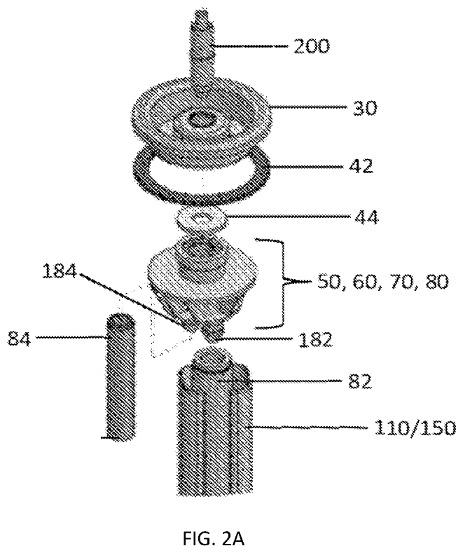

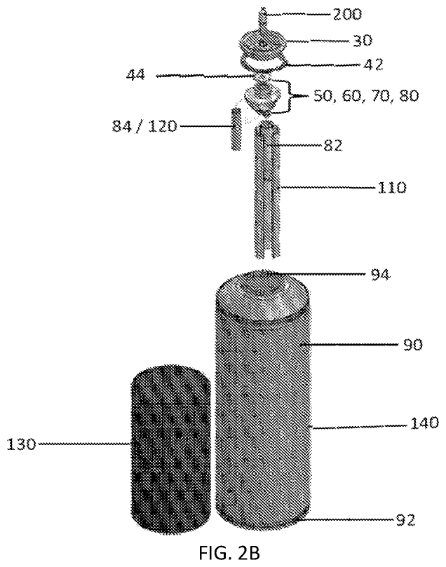

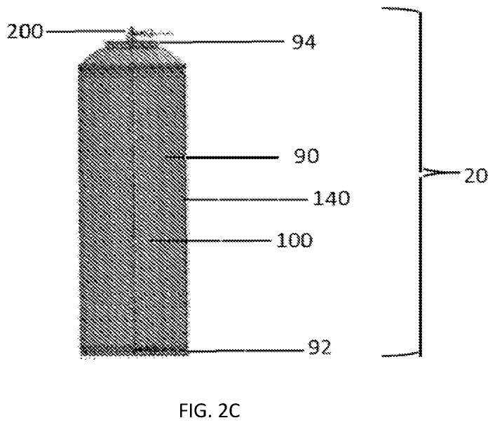

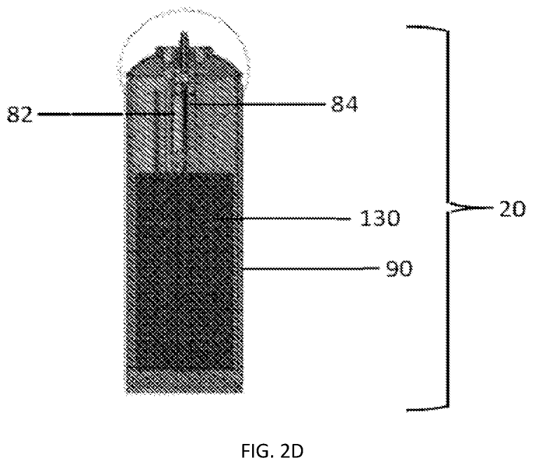

In other examples, like those shown in FIGS. 2B-D, the aerosol container can have a dispensing system in which two bags are filled, allowing two different products to be dispensed, either as separate products, or more typically as a single product, with mixing occurring in the valve assembly or actuator. In the latter case the valve assembly can have a dip tube (80) which splits/bifurcates into two, each with fitments for connecting a bag thereto. The bags are typically 3-layer or 4-layer pouches made respectively of polyacrylate/aluminum/polypropylene or polyethylene (PA/ALU/PP or PE) or polyethylene terephthalate/aluminum/orientated polyamide/polypropylene or polyethylene (PET/ALU/OPAi PP or PE).

In some examples, as illustrated in FIGS. 2A and 2B, the valve assembly (10) can have a mounting cup (30), a pair of gaskets (42 and 44), a valve seat (50), spring (60) and housing (70), with a dip tube (80) which divides, at its lower end, to receive two tubes (82; 84) on respective fitments (182; 184). An ingredient (100) containing reservoir (110), which can be a rigid sided container, bag or pouch can be connected to the first tube (82). First tube (82) and second tube (84) can extend into spray device container (90), which is filled or partially filled with a dispensing carrier gas (140), typically a compressed gas, like carbon dioxide or nitrogen. Optionally, the second tube (84) can acts to prevent fine particles of activated carbon (130) from being dispensed. Activated carbon (130) can adsorb the dispensing carrier gas which fills or partially fills the spray device (90). On actuation, the dispensing carrier gas (140) is released together with the ingredient (100) stored in containing reservoir (110), and the ingredient (100) and carrier gas (140) mix as they pass through the valve assembly (10) or actuator (200) to exit the spray device container via the actuator spray nozzle (200).

The spray device (20) illustrated in FIG. 2D, comprises a spray device container or cannister (90) (FIG. 2B) which is filled or partially filled with activated carbon (130) and the valve assembly (10) is crimped, or otherwise sealed, to close the opening (94) (FIG. 2B) of the dispensing cannister (90). The spray device (20) may be charged with a dispensing carrier gas (140) before or after crimping or otherwise sealing. Similarly, if the containing reservoir (110) is a bag or pouch it may be filled with its ingredients (100) before or after crimping.

An example of the filled spray device (20) is illustrated in FIGS. 2C and 2D.

The antiperspirant or deodorant product (100) can be in the form of a liquid or oil but could be any mobile phase. The antiperspirant or deodorant product (100) can be a stable single phase or it can be more than one phase.

If the containing reservoir (110) is made of a flexible material, such as a bag or pouch, it can be rolled into a hollow cylinder (see FIG. 2B) around first tube (82) for ease of insertion, and the adjoining second tube (84) can be inserted directly into the dispensing cannister or into a canister pre-filled with granular activated carbon (130), first and second tubes (82) and (84) being connected to the valve assembly via connectors (182) and (184) respectively. (If present, the granular carbon can easily displaced to accommodate the rolled-up bag which is now surrounded by the activated carbon granules). The canister is then crimped, and the bag side of the canister is filled with the required quantity of antiperspirant or deodorant product (100). The canister is then filled with pressurized gas (e.g. air, oxygen, nitrogen or carbon dioxide). On actuating the valve, the assembly enables the dispensing carrier gas (140), that is mixed or physically saturated, at least in part, with antiperspirant or deodorant product. See WO2020/021473, incorporated by reference, for additional examples of dispensers with bifurcated dip tubes where the pressurized gas and the product mix in the valve assembly before exiting the dispenser via the actuator spray nozzle.

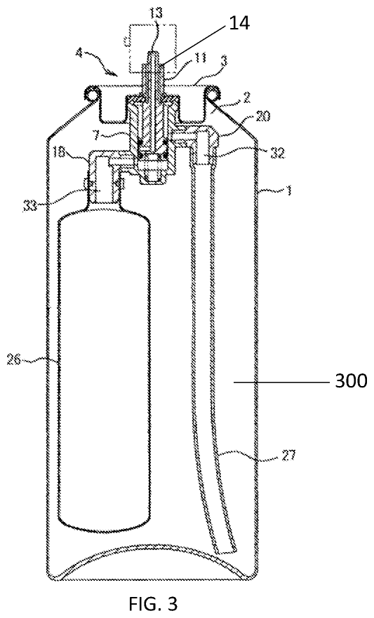

FIG. 3 is another example of an aerosol dispenser for antiperspirant or deodorant products. In this example, the pressurized gas (300) is directly filled in the aerosol container (1). Containing reservoir (26) can be filled with the antiperspirant or deodorant product. The containing reservoir (26) can be easily deformed with the pressurized gas. Further, the pressurized gas is connected to the second passage (14) disposed at the stem (11), via a dip tube (27), while the containing reservoir (26) is connected to the first passage (13) disposed at the stem (11).

In one example, the second passage (14) and first passage (13) are formed separately up to the top end of the stem (11) having a lower end thereof arranged inside the housing (7), while a second seal member seals the second liquid and the second passage (14) when the stem (11) is not pressed. In another example, the second the second passage (14) and first passage (13) are formed separately up to the valve assembly or actuator and the antiperspirant or deodorant composition combines with the pressurized gas before exiting the dispenser. See U.S. Pat. No. 7,798,366, incorporated by reference, for additional examples of an aerosol dispensing device with a valve assembly that can mix the product with the pressurized air in the valve assembly or the actuator immediately before dispensing.

### Spray Properties

It can be desirable for the spray device to have a total mass flow rate of the propellant/antiperspirant product mixture of less than 0.5 grams/sec, alternatively less than 0.4 grams/sec, alternatively less than 0.3 grams/sec, alternatively less than 0.25 g/sec, and alternatively less than or equal to 0.2 grams/sec. It can be desirable for the spray device to have a total mass flow rate of the propellant/antiperspirant product mixture of from about 0.05 grams/sec to about 0.6 grams/sec, or from about 0.1 grams/sec to about 0.4 grams/sec, or from about 0.15 grams/sec to about 0.3 grams/sec, or from about 0.2 grams/sec to about 0.25 grams/sec.

The amount of antiperspirant or deodorant product delivered to a target surface, such as the underarm, by a two second application from a spray device may be from about 0.1 g to about 1 g, alternatively from about 0.2 g to about 0.6 g, alternatively from about 0.3 g to about 0.5 g, alternatively about 0.2 g.

The distance the antiperspirant or deodorant product travels at least 6 in (15.24 cm), at least 8 in (20.32 cm), at least 10 in (25.4 cm), at least 12 in (30.48 cm), at least 14 in (35.56 cm), at least 16 in (40.64 cm), at least 18 in (45.72 cm).

The surface area the antiperspirant or deodorant product covers at a distance of 18 in (45.72 cm) is from about 2 in² (12.9 cm²) to about 79 in² (509.7 cm²), alternatively from about 10 in² (64.5 cm²) to about 50 in² (322.6 cm²), alternatively from about 20 in² (129 cm²) to about 40 in² (258 cm²), and alternatively from about 25 in² (161.3 cm²) to about 35 in² (225.8 cm²).

The antiperspirant or deodorant composition can be sprayed as the ejected composition. The ejected composition comprises/consists of particles having an average particle size distribution (Dv50) of at least 25 micron, alternatively at least 35 micron. The average particle size distribution (Dv50) is important in view of ejected composition drying time, which must be consumer acceptable. Indeed, a smaller average particle size distribution (Dv50) is useful in that more particles have a higher surface area to volume ratio, which means a faster drying time. On the other hand, a too low average particle size distribution (Dv50) means that not enough antiperspirant or deodorant composition is applied to underarms. A Malvern Spraytec instrument is used to measure the particle size distribution. The Dv50 is the term to describe the maximum particle size diameter below which 50% of the sample volume possesses, also known as the median particle size by volume. The Malvern Spraytec instrument uses the technique of laser diffraction for measurement of the size of the spray particles. The intensity of light scattered as a laser beam passes through a spray is measured. This data is then analysed to calculate the size of the particles that created the scattering pattern. A Malvern Spraytec 2000 is used according to the manufacturer's instructions. Test samples have a temperature between 20° C. to 22° C.

The ejected composition can have an average particle size distribution (Dv50) of less than 50 microns. The ejected composition can have an average particle size distribution (Dv50) of from about 15 micron, or from about 25 micron, or from about 30 micron, or from about 35 micron, or from about 40 micron, to about 70 micron, or to about 60 micron, or to about 55 micron, or to about 50 micron.

The total mass flow rate, average particle size distribution, distance sprayed, and product coverage can vary less than 50%, alternatively less than 40%, alternatively less than 30%, alternatively less than 25%, alternatively less than 20%, alternatively less than 15%, alternatively less than 10%, from the mean value for the first 25% of the can vs the mean value for the last 25% of the composition. Furthermore, the composition can be dispensed without substantial clogging in the spray device flow path from the first spray to the last spray.

### Propellant

The propellant may have a concentration from about 30%, 32%, 34% 36%, 38%, 40%, or 42% to about 70%, 65%, 60%, 58%, 56%, 54%, 52%, 50%, 48%, 46%, 44%, or 42% by weight of the total fill of materials (i.e., propellant and deodorant or antiperspirant composition) stored within the spray device. The volume of the propellant stored within the container may be from about 10 mL, 20 mL, 30 mL, or 40 mL to about, 80 mL, 70 mL, 60 mL, or 50 mL. In another example, the volume of liquid propellant stored within the container may be from about 81 mL, 90 mL, 100 mL, 120 mL, 140 mL or 140 mL to about 225 mL, 200 mL, 180 mL, 170 mL, 160 mL, or 150 mL.

Propellant pressure may affect the mass flow and/or spray characteristics of the antiperspirant composition/liquid propellant mixture. It is believed that when the propellant concentration is too low, the total fill of the container may result in too high of a mass flow of the antiperspirant composition and/or poor spray characteristics (i.e. a narrow spray pattern).

Deodorant and/or Antiperspirant Compositions

The term “antiperspirant composition” refers to any composition containing a deodorant or antiperspirant active and which is intended to be applied onto skin. The term “deodorant composition” refers to any composition containing a deodorant active and which is intended to be applied onto skin.

The term “aerosol antiperspirant composition” refers to a deodorant or antiperspirant composition that is pressurized and/or atomized by a propellant.

The term “aerosol spray device” refers to a spray device that uses a propellant to pressurize a deodorant or antiperspirant composition and/or atomize a deodorant or antiperspirant composition when sprayed.

The term “activated” refers to a clay material which has undergone a volume increase.

The term “antiperspirant efficacy” refers to the amount of wetness protection provided by application of a deodorant or antiperspirant composition to an underarm area (or axillia) by a spray device. Antiperspirant efficacy may be quantified by the amount (mg) of sweat collected following exposure to a hot room compared to a baseline amount.

The term “at the time of making” refers to a characteristic (e.g., viscosity) of a raw material ingredient just prior to mixing with other ingredients.

The term “bulking or suspending material” refers to a material which is intended to reduce settling of a particulate from a liquid and/or reduce the severity of particulate caking post settling.

The terms “clay” and “clay material” refer generally to a variety of: i) clay minerals, including but not limited to the following groups: kaolin (e.g., kaolinite, dickite, halloysite, and nacrite), smectites (e.g., montmorillonite, bentonite, nontronite, hectorite, saponite and sauconite), illites and chlorites; and ii) organoclay materials.

The term “clay activator” refers to a polar material which increases the volume fraction of the clay material and/or the viscosity or yield point of the antiperspirant composition.

The term “clogging” refers to: i) either a blocked passage, orifice, hole or other opening resulting in little or no mass flow out of a container when the actuator is activated, or ii) a valve stuck at least partially open from accumulated composition, resulting in semi-continuous or continuous leakage of the antiperspirant composition and/or a propellant from the spray device, or iii) accumulation of antiperspirant composition within a portion of the flow path of the container which substantially impacts performance of the spray device.

The term “container” and derivatives thereof refers to the package that is intended to store and dispense a deodorant or antiperspirant composition in a spray type form. A container may typically comprise a reservoir for storing the antiperspirant composition, a valve for controlling flow of the antiperspirant composition, and an actuator by which a user can actuate the valve.

The term “deposition efficiency” refers to the percentage of a material (e.g., antiperspirant active, fragrance material, antiperspirant composition, etc.) that is deposited on a target surface compared to the amount of material that exits in a spray device.

The term “particulate”, as used herein, refers to a material that is solid or hollow or porous (or a combination thereof) and which is substantially or completely insoluble in the liquid materials of a deodorant or antiperspirant composition.

The term “substantially free of” refers to an amount of a material that is less than 1%, 0.5%, 0.25%, 0.1%, 0.05%, 0.01%, or 0.001% by weight of a deodorant or antiperspirant composition. “Free of” refers to no detectable amount of the stated ingredient or thing.

The term “total fill” or “total fill of materials” refers to the total amount of materials added to or stored within a reservoir(s) of a container. For example, total fill includes the propellant and antiperspirant or deodorant composition stored within a device after completion of filling and prior to first use.

The term “viscosity” means dynamic viscosity (measured in centipoise, cPs, or Pascal-second, Pa·s) or kinematic viscosity (measured in centistokes, cst, or m²/s) of a liquid at approximately 25° C. and ambient conditions. Dynamic viscosity may be measured using a rotational viscometer, such as a Brookfield Dial Reading Viscometer Model 1-2 RVT available from Brookfield Engineering Laboratories (USA) or other substitutable model known in the art. Typical Brookfield spindles which may be used include, without limitation, RV-7 at a spindle speed of 20 rpm, recognizing that the exact spindle may be selected as needed by one skilled in the art. Kinematic viscosity may be determined by dividing dynamic viscosity by the density of the liquid (at 25° C. and ambient conditions), as known in the art.

In all embodiments of the present invention, all percentages are by weight of the antiperspirant or deodorant composition (or formulation), unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. The term “molecular weight” or “M.Wt.” as used herein refers to the number average molecular weight unless otherwise stated.

Viscosity

In some embodiments, it may be desirable for the viscosity of the deodorant or antiperspirant composition to be from about 1,000 centipoise, 2,000 centipoise, or 3,000 centipoise to about 50,000 centipoise 40,000 centipoise, or 30,000 centipoise, or 20,000 centipoise, or 10,000 centipoise, or 5,000 centipoise or 4,000 centipoise at 25° C. (1 centipose being equal to 1×10⁻³ Pa·s). It is believed that a viscosity lower than 1,000 centipoise may lead to a deodorant or antiperspirant composition, which when spayed, results in a runny or drippy effect on skin. This may be perceived by a user as having a wet rather than dry feel. The deodorant or antiperspirant compositions described herein can be flowable so that it may be sprayed effectively from a spray device, the antiperspirant composition may be devoid of ingredients in sufficient concentrations that provide a deodorant or antiperspirant stick-type rheology. Some common agents which may be excluded in meaningful amounts include hydrogenated castor oil, solid paraffins, silicone waxes, and mixtures thereof

Non-Volatile Silicone Fluids

The deodorant or antiperspirant compositions can comprise one or more non-volatile silicone fluids. The non-volatile silicone fluid may function as the primary or principal liquid carrier for the antiperspirant active. As used herein, the term “non-volatile” refers to a material that has a boiling point above 250° C. (at atmospheric pressure) and/or a vapor pressure below 0.1 mm Hg at 25° C. Conversely, the term “volatile” refers to a material that has a boiling point less than 250° C. (at atmospheric pressure) and/or a vapor pressure about 0.1 mm Hg at 25° C.

The total concentration of non-volatile, silicone fluids may be from about 30%, 35%, 40%, 45%, 50% to about 70%, 65%, 60%, 55% or 50% by weight of a deodorant or antiperspirant composition. In some embodiments, the total concentration of non-volatile, silicone fluids may be from about 35% to about 55% by weight of a deodorant or antiperspirant composition.

The liquid materials of the antiperspirant composition may consist essentially of or primarily comprise a non-volatile, silicone fluid(s). Some non-volatile, silicone fluids that may be used include, but are not limited to, polyalkyl siloxanes, polyalkylaryl siloxanes, and polyether siloxane copolymers, and mixtures thereof. Some preferred non-volatile silicone fluids may be linear polyalkyl siloxanes, especially polydimethyl siloxanes (e.g., dimethicone). These siloxanes are available, for example, from Momentive Performance Materials, Inc. (Ohio, USA) under the tradename Element 14 PDMS (viscosity oil). Silicones Fluids from Dow Corning Corporation (Midland, Mich., USA) available under the trade name Dow Corning 200 Fluid series (e.g., 3 to 350 centistokes). Other non-volatile silicone fluids that can be used include polymethylphenylsiloxanes. These siloxanes are available, for example, from the General Electric Company as SF 1075 methyl phenyl fluid or from Dow Corning as 556 Fluid. A polyether siloxane copolymer that may be used is, for example, a dimethyl polyoxyalkylene ether copolymer fluid. Such copolymers are available, for example, from the General Electric Company as SF-1066 organosilicone surfactant. The non-volatile, silicone fluid may have an average viscosity from about 3 centistokes, 5 centistokes, 10 centistokes, 20 centistokes, or 50 centistokes to about 350 centistokes, 200 centistokes, 100 centistokes, 50 or 30 centistokes at 25° C. (1 centistoke being equal to 1×10⁻⁶ m²/s). In some specific embodiments, the silicone fluid may have a viscosity from about 5 centistokes to about 100 centistokes or 5 centistokes to about 50 centistokes or about 5 centistokes to about 30 centistokes. In some instances, the non-volatile silicone fluid is a polydimethylsiloxane fluid (also commonly referred to as dimethicone). It will be appreciated that a polydimethylsiloxane fluid may be further characterized by, optionally, its viscosity or its molecular weight or its formula or a combination thereof. In some instances, the polydimethylsiloxane fluid may have the following characteristics:

TABLE 1

| | | Approximate |
|---|---|---|
| | | Average |
| | Approximate | Number of |
| | Molecular | Monomer Units |
| Viscosity | Weight¹ | in the Polymer¹ |
| 3 Centistokes | 500 | 6 |
| 5 Centistokes | 800 | 9 |
| 10 Centistokes | 1200 | 13 |
| 20 Centistokes | 2000 | 27 |
| 30 Centistokes | 2600 | 35 |
| 50 Centistokes | 3800 | 50 |
| 100 Centistokes | 6000 | 80 |
| 200 Centistokes | 9400 | 125 |
| 350 Centistokes | 13,700 | 185 |

¹The compositions of Examples 1 to 14, to the extent they contained a dimethicone fluid, were formulated utilitizing a Dow Corning DC200 series fluid, which is believed to have had average molecule weights and average number of monomer subunits falling within the approcximate values of above-described table.

The polydimethylsiloxane fluid may have the following formula (II):

M-D_{X}-M

wherein M is (CH₃)₃SiO and D is 2CH₃(SiO) and X is equal to the average number of monomer units (see, e.g., Table 1) in the polymer minus 2. In some embodiments, X may be from about 6 to about 185, from about 9 to about 125, from about 9 to about 80, from about 9 to about 50, from about 13 to about 50 or from about 27 to about 50. In other embodiments, X may be from about 6 to about 35, from about 9 to about 35 or from about 13 to about 35. The term “approximate” as used in Table 1 refers to ±10% of a given value.

Liquid Fragrance Materials

A deodorant or antiperspirant composition may also optionally comprise one or more liquid fragrance materials. Liquid fragrance materials are typically a mixture of perfume or aromatic components that are optionally mixed with a suitable solvent, diluent or carrier. Some or many of the perfume components, when combined, may result in a highly polar liquid fragrance material. Some suitable solvents, diluents or carriers for the perfume components may include ethanol, isopropanol, diethylene glycol monoethyl ether, dipropylene glycol, diethyl phthalate, triethyl citrate, isopropyl myristate and mixtures thereof. A deodorant or antiperspirant composition may comprise from about 2%, 3% or 4% to about 10%, 8%, 6%, or 4% by weight of a liquid fragrance material.

Without intending to be bound by any theory, it is believed that, in some instances, a liquid fragrance concentration less than about 2% by weight of the antiperspirant composition may not deliver sufficient long lasting scent throughout the day For example, in some instances, it may be desirable for the fragrance to last greater than 8 hrs, 10 hrs, 12 hrs, 14 hrs or 16 hrs. Furthermore, a fragrance level less than about 4% may be less desirable for providing a long lasting scent experience in deodorant or antiperspirant compositions comprising a non-volatile silicone fluid and propellant concentration more than 71% by weight of the total fill of materials.

The perfume component may be any natural or synthetic perfume component known to one skilled in the art of creating fragrances including, but not limited to, essential oils, citrus oils, absolutes, resinoids, resins, concretes, etc., and synthetic perfume components such as hydrocarbons, alcohols, aldehydes, ketones, ethers, acids, esters, acetals, ketals, nitriles, etc., including saturated and unsaturated compounds, aliphatic, carbocyclic and heterocyclic compounds. Some non-limiting examples of perfume components include: geraniol, geranyl acetate, linalool, linalyl acetate, tetrahydrolinalool, citronellol, citronellyl acetate, dihydromyrcenol, dihydromyrcenyl acetate, tetrahydromyrcenol, terpineol, terpinyl acetate, nopol, nopyl acetate, 2-phenylethanol, 2-phenylethyl acetate, benzyl alcohol, benzyl acetate, benzyl salicylate, benzyl benzoate, styrallyl acetate, amyl salicylate, dimethylbenzyl carbinol, trichloromethylphenyl-carbinyl acetate, p-tert.butyl-cyclohexyl acetate, isononyl acetate, vetiveryl acetate, vetiverol, alpha-n-amylcinammic aldehyde, alpha-hexylcinammic aldehyde, 2-methyl-3-(p-tert.butylphenyl)-propanol, 2-methyl-3-(p-isopropylphenyl)-propanal, 3-(p-tert.butylphenyl)-propanal, tricyclodecenyl acetate, tricyclodecenyl propionate, 4-(4-hydroxy-4-methylpentyl)-3-cyclohexene carbaldehyde, 4-(4-methyl-3-pentenyl)-3-cyclohexene carbaldehyde, 4-acetoxy-3-pentyltetrahydropyran, methyldihydrojasmonate, 2-n-heptylcyclopentanone, 3-methyl-2-pentylcyclopentanone, n-decanal, 9-decenol-1, phenoxyethyl isobutyrate, phenyl-acetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepine nitrile, aubepine, heliotropine, coumarin, eugenol, vanillin, diphenyl oxide, hydroxycitronellal, ionones, methylionones, isomethylionones, irones, cis-3-hexenol and esters thereof, indane musk fragrances, tetralin musk fragrances, isochroman musk fragrances, macrocyclic ketones, macrolactone musk fragrances, ethylene brassylate, aromatic nitro-musk fragrances. Some perfume components are also described in Arctander, Perfume and Flavour Chemicals (Chemicals), Vol. I and II (1969) and Arctander, Perfume and Flavour Materials of Natural Origin (1960).

Clay Materials and Clay Activators

A deodorant or antiperspirant composition comprises a clay material as a bulking or suspending agent. The concentration of clay material may be from about 1%, 2%, 3% to about 8%, 6%, 5%, or 4% by weight of the antiperspirant composition. In some embodiments, the concentration of the clay material is from about 2% to about 6% by weight of the antiperspirant composition. In some embodiments, the total particulates of antiperspirant composition may comprise from about 5% to about 20% or 5% to 15% of a clay material. In some embodiments clay materials are organoclays, which may be derived from clay minerals in which a portion of the inorganic cationic counter ions (e.g., sodium cations) of the clay mineral have been exchanged for organocations (e.g., quaternary ammonium chloride) thereby rendering the material organophilic rather than hydrophilic. Shearing/milling of the clay material deagglomerates the clay material platelets after which a polar clay activator may be added in some instances to further separate the platelets and promote the formation of hydrogen bonds between the edges of adjacent platelets. This enables formation of a higher volume three dimensional clay structure that suspends the particulates of the antiperspirant composition. This also increases the volume of the clay material in the antiperspirant composition, thereby increasing the volume or bulk of the total powder of the antiperspirant composition. This is also why the settling height of a deodorant or antiperspirant composition may be one quantitative/qualitative measure of the amount/quality of activation of a clay material.

Some non-limiting examples of clay materials include montmorillonite clays and hydrophobically treated montmorillonite clays. Montmorillonite clays are those which contain the mineral montmorillonite and may be characterized by a having a suspending lattice. Some examples of these clays include but are not limited to bentonites, hectorites, and colloidal magnesium aluminum silicates. Some non-limiting examples of organoclays include modified bentonite, modified hectorite, modified montorlinite and combinations thereof, some examples of which are available under the trade names Bentone 27 (stearalkonium bentonite), Bentone 34 (stearalkonium bentonite) and Bentone 38 (disteardimonium hectorite) from Elementis Specialities Plc. and Tixogel VPV (quaternium 90-bentonite), Tixogel VZV (stearalkonium bentonite), Tixogel LGM (stearalkonium bentonite) and Claytone SO (stearalkonium bentonite) from Southern Clay Products. In some instances, the bulking and suspending material consists substantially of, essentially of and/or primarily of a clay material and more preferably an organoclay material. In these instances, the antiperspirant composition may be substantially or completely free of silica materials used as a bulking/suspending material.

The antiperspirant composition may also comprise a clay activator, such as propylene carbonate, triethyl citrate, methanol, ethanol, acetone, water and mixtures and derivatives thereof. Without intending to be bound by any theory, it is believed that the clay activator enhances the hydrogen bonds between the edges of adjacent clay platelets. Too little clay activator may provide insufficient hydrogen bonding between clay platelets while too much may create very strong interactions resulting in formation of agglomerates and loss of the desired bulking benefit. The clay activator may have a concentration ranging from 1:3 to 2:3 parts clay activator to clay material. Clay activators may also strongly interact with a deodorant or antiperspirant active (e.g., leading to clumping or coating of the antiperspirant active and/or changes in active polymer structure which may reduce antiperspirant efficacy). Therefore, it may be desirable to limit the amount of clay activator present in the antiperspirant composition to between about 0.5%, 0.75%, 1%, 1.25%, or 1.5% to about 3%, 2%, or 1.75% by weight of the antiperspirant composition.

Liquid Activation Enhancer

Without intending to be bound by any theory, it is believed that certain liquid materials may help maintain and/or promote the clay bulking and suspending benefit in a deodorant or antiperspirant composition that comprises a non-volatile silicone liquid, and optionally a liquid fragrance material, by facilitating increased interaction or loose bonding between the non-volatile silicone fluid and the clay material. It is believed that the increased interaction may be facilitated, in some instances, when the liquid activation enhancer is soluble in the non-volatile silicone and has a Hansen Solubility Parameter for Hydrogen Bonding, δₕ, between about 2 MPa^{1/2} and about 6 MPa^{1/2}.

Liquid activation enhancers that are soluble in the non-volatile silicone fluid may advantageously: 1) disperse within the non-volatile silicone fluid, thereby promoting a more uniform interaction or loose bonding between the clay material and the non-volatile silicone fluid, and/or 2) minimize regions of high clay activation by increasing the solubility and/or dispersability of the clay activator and/or optional liquid fragrance material, thereby reducing the risk of locally high concentrations of the clay activator and/or liquid fragrance material which may result in clay precipitation. Solubility may be determined by measuring the amount of light transmittance (a light transmittance value) through a simple mixture of the non-volatile silicone fluid and liquid activation enhancer at the same weight/weight concentrations as in a final antiperspirant composition. For example, the solubility of a liquid activation enhancer at a concentration of 9% w/w in a final antiperspirant composition comprising a non-volatile silicone fluid having a concentration of 38% w/w can be determined by measuring the light transmittance of a simple mixture of the liquid activation enhancer at 19% w/w concentration in just the non-volatile silicone fluid. Light transmittance may be measured using a spectrophotometer, such as, for example, a Spectronic Genesys 10 Vis Spectrophotometer available from Thermo Electron Corp (USA), wherein a light transmittance value greater than 80%, 85%, 90% or 95% at 25° C. indicates sufficient solubility in the non-volatile silicone fluid.

It is also believed that a liquid activation enhancer having a δₕ value between 2 MPa^{1/2} and 6 MPa^{1/2} may also promote interaction or loose bonding between non-volatile silicone fluid and the clay material. It is believed that δₕ values less than about 2 MPa^{1/2} may be insufficient to provide adequate interaction or loose bonding between the non-volatile silicone fluid and the clay material while values greater than about 6 may result in collapse of the three dimensional clay structure due to the creation of strong hydrogen bonding between the clay platelets. In some instances, it may also be desirable that the liquid activation enhancer is also capable of solubilizing both the liquid fragrance material and the clay activator in order to avoid regions of high/low clay activation, as these materials may not be easily solubilized in non-volatile silicone fluids.

A deodorant or antiperspirant composition comprises at least one liquid activation enhancer. The at least one liquid activation enhancer, or the combination of a plurality of activation enhancers, may have a total concentration from about 2%, 4%, 6%, 8%, 10% to about 30%, 25%, 20%, 18%, 16%, 14%, 12%, 10% or 8% by weight of the antiperspirant composition. In some embodiments, the liquid activation enhancer has a concentration from about 2% to about 15% by weight of the antiperspirant composition. It is believed that concentrations higher than 30% may impact spreading of the antiperspirant composition on skin by increasing the surface tension of the composition, which is one mechanism by which a dry skin feel may be imparted in a deodorant or antiperspirant composition comprising a non-volatile silicone fluid. It also believed that concentrations less than 2% may be too low to provide sufficient interaction between the clay material and the non-volatile silicone fluid

Some liquid activation enhancers can be molecules comprising a fatty or hydrocarbon group and a functional group that is capable of hydrogen bonding near or at one terminus of the hydrocarbon group. The hydrocarbon chain may be from about 8 to about 20 carbon atoms in length (C₈ to C₂₀) to provide the desired solubility in the non-volatile silicone fluid. The hydrocarbon chain may be linear, branched, unbranched, saturated or unsaturated. The hydrogen bonding group may be selected from the group consisting of alcohol, ester, amide and aryl/aromatic groups. Most preferred are hydrogen bonding accepting groups such as esters and aromatic groups. Some non-limiting examples of these materials include esters and amides formed from the reaction of fatty acids, fatty amines, or fatty alcohols with alcohols, amines, or carboxylic acids. Some non-limiting examples of fatty acids, fatty amines, and fatty alcohols include stearic acid, palmitic acid, myristic acid, lauric acid, stearyl amine, palmityl amine, myristyl amine, stearyl alcohol, palmityl alcohol, myristyl alcohol and lauryl alcohol. Some non-limiting examples of alcohols, amines, or carboxylic acids include, methanol, ethanol, propanol, isopropanol, butanol, isobutanol, phenyl alcohol, benzyl alcohol, phenol, methyl amine, ethyl amine, propyl amine, butyl amine, benzyl amine, formic acid, acetic acid, propanoic acid, butyric acid and benzoic acid.

Some non-limiting examples of liquid activation enhancers can include isopropyl myristate, isopropyl palmitate, ethyl stearate, methyl stearate, propyl stearate, butyl stearate, ethyl myristate, ethyl palmitate, butyl palmitate, propyl stearate, propyl palmitate, methyl stearamide, ethyl stearamide, isopropyl stearamide, ethyl palmitamide propyl palmitamide, stearyl benzoate, palmityl benzoate, C12-15 alkyl benzoate, benzyl palmitate, benzyl stearate, dodecylenbenezene and palmityl acetate. Liquid activation enhancers might also include fatty branched chain alcohols and ethoxylated fatty alcohols. The liquid activation enhancer may have the following formula (I):

R₁—X—R₂

wherein R₁ contains from about 8 to about 20 carbon atoms, X is selected from the group consisting of alcohol, ester, amide and aryl groups, and R₂ is selected from the group consisting of null, hydrogen (H), 1 to 4 carbon atoms, and C₆H₆.

Some particularly preferred non-limiting examples of liquid activation enhancers suitable for use include isopropyl myristate (δₕ=about 2.95, light transmittance values about 101% at concentrations from 2% to 30% w/w in 50 centistoke dimethicone), isopropyl palmitate (δₕ=about 3.15, light transmittance values about 101% at concentrations from 2% to 30% w/w in 50 centistoke dimethicone), butyl stearate (δₕ=about 3.45, light transmittance values about 100% at concentrations from 2% to 30% w/w in 50 centistoke dimethicone) and, in some instances, C12-15 alkyl benzoate (available under the trade name Finsolv® from Innospec Performance Chemicals, USA) and combinations thereof.

Some liquid materials may have a δₕ between 2 and 6 and straddle the line between soluble and not soluble in the non-volatile silicone fluid, depending on the w/w concentration of the material in the non-volatile silicone fluid and/or the viscosity/molecular weight of the non-volatile silicone fluid. One such material is C12-15 alkyl benzoate (δₕ=about 4.7), available under the trade name Finsolv®. C12-15 alkyl benzoate has light transmittance values of about 101%, about 102%, about 1.4% and about 0.2% at concentrations of 2%, 9%, 15% and 30% w/w, respectively, in 50 centistoke dimethicone.

In some instances, the liquid activation enhancer may also sufficiently activate the organoclay material without the need for a separate clay activator, such as propylene carbonate, triethyl citrate, methanol, ethanol, acetone and mixtures and derivatives thereof. A non-limiting example of one such material is C12-15 alkyl benzoate. Referring to Examples 21 and 22, two antiperspirant composition comprised, in part, 20 centistoke dimethicone and C12-15 alkyl benzoate (9% w/w). The antiperspirant composition of Example 21 comprised triethyl citrate and the antiperspirant composition of Example 22 did not. Both antiperspirant compositions had a powdery redispersion, indicating that the organoclay material was activated in both.

It is also believed that the viscosity of the non-volatile silicone fluid may in some instances impact the solubility of the liquid activation enhancer in the non-volatile silicone fluid. In some embodiments, the viscosity of the non-volatile silicone fluid is from about 3 centistokes, 5 centistokes, 10 centistokes, 15 centistokes, 20 centistokes, 50 centistokes and 100 centistokes to about 350 centistokes, 200 centistokes, 100 centistokes or 50 centistokes. Preferably, the viscosity of the non-volatile silicone fluid is from about 5 centistokes to about 100 centistokes, more preferably between about 5 centistokes and about 50 centistokes. In some embodiments, the non-volatile silicone fluid has a viscosity from about 5 centistokes to about 30 centistokes.

Since both a non-volatile silicone fluid and a liquid fragrance material may negatively affect clay activation, it is believed that the at least one liquid activation enhancer may be most beneficial in those instances where the concentration of the liquid fragrance material exceeds the concentration of the clay material and/or where the concentration of the liquid fragrance material exceeds the concentration of the clay activator. In some embodiments, the ratio of total concentration of non-volatile silicone fluid to the total concentration of liquid activation enhancer is from about 2:1 to about 10:1, or about 3:1 to about 5:1.

Order of Addition of the Liquid Fragrance Materials and Non-Volatile Silicone Fluid

It is believed that the clay activation and desired bulking benefit may be optionally further improved by controlling the order of addition of the liquid fragrance material and/or the clay material in the making of a deodorant or antiperspirant composition, particularly at liquid fragrance concentrations greater than 2% by weight of the antiperspirant composition. Without intending to be bound by any theory, it is believed that managing how the liquid fragrance material (particularly those that are highly polar) is added/solubilized may reduce regions of high strong interaction between the liquid fragrance material and the clay material that are believed to result in agglomeration of the clay material and/or precipitation thereof. In one example, a making process for a deodorant or antiperspirant composition may comprise a plurality of steps. The first step can comprise optionally mixing a first portion of the non-volatile silicone fluid (e.g., 10% to 30% of the total concentration of the final antiperspirant composition) with the clay material and the liquid activation enhancer. The second step can comprise adding a clay activator to the mixture of the first step. It will be appreciated that, in some instances, a clay activator may not be needed, and this step may be skipped. This can be followed by adding a second portion of the non-volatile silicone fluid in a third step, after which the particulates are added in a fourth step to form a first composition. In this embodiment, the first composition is then ready to be filled into an ingredient container.

Particulate Materials

Delivering a sufficient concentration of particulates to the skin is believed to improve the skin feel of a deodorant or antiperspirant composition comprising a high concentration of a non-volatile silicone fluid. It is believed that a deodorant or antiperspirant composition comprising a total non-volatile liquid material to total particulate material ratio (L/P ratio) from about 0.6, 0.8, 1, 1.2, or 1.4 to about 2.3, 2.2, 2.1, 2, 1.9, 1.8 or 1.6 may balance the tradeoff between enough particulates to provide acceptable skin feel while minimizing the appearance of residue. A deodorant or antiperspirant composition may have a total particulate concentration from about 30%, 35%, or 40% to about 60%, 55%, or 50% by weight of the antiperspirant composition, in keeping with the total liquid to total particulate (L/P) ratios previously described. While increasing the concentration of particulates may improve skin feel, it may also lead to an increased risk of clogging especially at low propellant concentrations.

The antiperspirant composition may comprise a variety of particulate materials. However, it is believed that the type (e.g., hydrophilic v. hydrophobic) and concentrations of particulate materials included in a deodorant or antiperspirant composition may, in some instances, impact skin feel, release of the antiperspirant active, and the propensity for clogging in the spray device. For example, too much antiperspirant active may result in a wet or sticky skin feel due to the propensity of antiperspirant actives to become sticky when hydrated (e.g., by perspiration) even within the L/P ratios previously described. In addition, too much of a hydrophobic particulate material may reduce release of the antiperspirant active from the composition. Conversely, inclusion of a hydrophilic particulate material may advantageously aid release of the antiperspirant active, which may be beneficial in a composition comprising a high concentration of a non-volatile silicone fluid. However, hydrophilic materials may increase the risk of clogging in the presence of water. Therefore, it may be desirable to balance these and other design considerations when incorporating particulate materials in a deodorant or antiperspirant composition comprising a non-volatile silicone fluid that is in turn used in a spray device especially those with low propellant concentration.

Some examples of particulate materials include, but are not limited to, antiperspirant actives, powders (e.g., starch materials), encapsulated fragrance materials and bulking or suspending agents (e.g., clay materials). Other types of particulates may also be incorporated in a deodorant or antiperspirant composition.

Antiperspirant Actives

A deodorant or antiperspirant composition can comprise one or more antiperspirant actives. The antiperspirant active may be any particle having antiperspirant activity. The antiperspirant active is preferably insoluble in the liquid components of the antiperspirant composition. Since the amount of antiperspirant active may significantly impact skin feel, a deodorant or antiperspirant composition may comprise from about 14% 16%, 18%, 20%, 22%, or 24% to about 38%, 36%, 34%, 32%, 30%, 28%, or 26% by weight of a particulate antiperspirant active. In some instances, it may be desirable to utilize a low concentration of the antiperspirant active, such as less than 20% or 18% by weight of the antiperspirant composition. The antiperspirant active concentrations refer to the anhydrous amount that is added. The antiperspirant active may represent the highest concentration of particulate materials in the antiperspirant composition. For example, the antiperspirant active (on an anhydrous basis) may comprise from about 50% to about 80%, or from about 50% to about 75%, or from about 55% to about 70% of the total particulate materials in the antiperspirant composition. The balance of the total particulate concentration comprises non-antiperspirant active particulates.

Some examples of suitable antiperspirant actives include astringent metallic salts, particularly including the inorganic and organic salts of aluminum. Some non-limiting examples exemplary aluminum salts that can be used include aluminum chloride and the aluminum hydroxyhalides having the general formula Al₂(OH)ₐQ_{b}XH₂O where Q is chloride, bromide, or iodide (preferably chloride), a is from about 2 to about 5, and a+b=about 6, and a and b do not need to be integers, and where X is from about 1 to about 6, and X does not need to be an integer. Particularly preferred are the aluminum chlorhydroxides referred to as “5/6 basic chlorhydroxide” wherein “a” is 5 and “2/3 basic chlorhydroxide” wherein “a” is 4. Aluminum salts of this type can be prepared in the manner described more fully in U.S. Pat. Nos. 3,887,692; 3,904,741; and 4,359,456. Preferred compounds include the 5/6 basic aluminum salts of the empirical formula Al₂(OH)₅DI2H₂O; mixtures of AICl₃6H₂O and Al₂(OH)₅Cl₂H₂O with aluminum chloride to aluminum hydroxychloride weight ratios of up to about 0.5.

The aluminum salt may be prepared by methods well known in the art. In some embodiments, the aluminum salts may be made by applying heat to a dilute aqueous solution of an aluminum salt (e.g., less than 20% of an aluminum salt by weight of the dilute solution) to form a solid aluminum salt comprising aluminum hydrolysis polymers. Some non-limiting examples of such methods are described in U.S. Pat. Nos. 4,871,525 and 4,359,456.

Substantially Inert Particulate Materials

The balance of the total particulate concentration of a deodorant or antiperspirant composition may comprise excipient particulate materials that are substantially inert with respect to the non-volatile silicone fluid. The excipient particulate materials may be either hydrophilic or hydrophobic (including hydrophobically modified, which tend to be moderately hydrophobic). Some non-limiting examples of substantially inert excipient particulate materials that may be included in a deodorant or antiperspirant composition include, but are not limited to, encapsulated fragrance materials; native starches such as tapioca, corn, oat, potato, and wheat starch particulates; talc; calcium carbonate; perlite; mica and polyethylene beads.

The substantially inert particulates may be free flowing. A deodorant or antiperspirant composition may comprise from about 0.25%, 0.5%, 1%, 5%, 10%, 12%, or 14% to about 25%, 22%, 20%, 18%, or 16% by weight of the antiperspirant composition of substantially inert particulates.

One substantially inert particulate material believed to be suitable for use is a hydrophilic or hydrophobically modified tapioca material. A tapioca material may be particularly beneficial as it is unlikely to induce an allergic reaction if inhaled. Tapioca is a starch which may be extracted from the cassava plant, typically from the root, which may then be processed or modified as known in the art. Tapioca starches are, advantageously, substantially non-allergenic. One non-limiting example of a hydrophobically modified tapioca material suitable for use comprises a silicone grafted tapioca starch, which is available under the trade name Dry Flo TS from AkzoNobel of the Netherlands. The INCI name is tapioca starch polymethylsilsesquioxane and may be produced by a reaction of methyl sodium siliconate (polymethylsilsesquioxane) and tapioca starch. This silicone grafted tapioca material is commercially available as CAS No. 68989-12-8. The silicone grafted tapioca material can be formed using any known means, including, but not limited to those methods described in U.S. Pat. Nos. 7,375,214, 7,799,909, 6,037,466, 2,852,404, 5,672,699, and 5,776,476. Other non-limiting examples of hydrophobically modified tapioca materials that are suitable for use include Dry Flo AF (silicone modified starch from Akzo Nobel), Rheoplus PC 541 (Siam Modified Starch), Acistar RT starch (available from Cargill) and Lorenz 325, Lorenz 326, and Lorenz 810 (available from Lorenz of Brazil). In some specific embodiments, the tapioca material may be hydrophilic in order to facilitate release of the antiperspirant active during use. One non-limiting example of a hydrophilic tapioca material suitable for use is available under the trade name Tapioca Pure available from Akzo Nobel. In some specific embodiments, the substantially inert particulate material comprises a hydrophilic tapioca material, a hydrophobic tapioca material or a mixture thereof.

A deodorant or antiperspirant composition may optionally comprise one or more particulate fragrance carriers. Fragrance carriers are typically particulates, which would be considered part of the total particulate concentration of the antiperspirant composition. The fragrance carriers are preferably hydrophobic in order to minimize particle-to-particle interactions. The fragrance carriers may be either full or empty. A full fragrance carrier is a fragrance carrier that encapsulates or otherwise contains a perfume component while the fragrance carrier is stored within the spray device. Full fragrance carriers may release their perfume components by a variety of mechanisms post delivery from the spray device to provide a desired aroma or fragrance experience for a user. For example, the perfume components may be released by moisture upon wetting of the fragrance carrier, e.g., by perspiration or other body fluids. Alternatively or in addition thereto, the perfume components may be released by fracture of the carrier, such as by the application of pressure or a shearing force. An empty fragrance carrier is a fragrance carrier that does not contain a perfume component while stored within the spray device. One non-limiting example of an empty fragrance carrier is an uncomplexed cyclodextrin material.

Some non-limiting examples of fragrance carriers suitable for encapsulating a perfume component include, but are not limited to, oligosaccharides (e.g., cyclodextrins), starches, polyethylenes, polyamides, polystyrenes, polyisoprenes, polycarbonates, polyesters, polyacrylates, vinyl polymers, silicas, and aluminosilicates. Some examples of fragrance carriers are described in USPNs 2010/0104611; 2010/0104613; 2010/0104612; 2011/0269658; 2011/0269657; 2011/0268802; U.S. Pat. Nos. 5,861,144; 5,711,941; 8,147,808; and 5,861,144.

A deodorant or antiperspirant composition may comprise from about 0.25%, 0.5%, 0.75%, 1%, or 2% to about 20%, 16%, 12%, 10%, 8%, 6% or 4% by weight of the antiperspirant composition of fragrance carriers. In some instances, the substantially inert excipient particles of the antiperspirant composition consist essentially of or completely of full fragrance carriers, empty fragrance carrier, or mixtures thereof. A deodorant or antiperspirant may comprise from about 0.25%, 0.5%, 0.75%, or 1% to about 6%, 4% or 2% by weight of the antiperspirant composition of full fragrance carriers. A deodorant or antiperspirant composition may comprise from about 0.25%, 0.5%, 1%, or 2% to about 16%, 12%, 10%, 8%, 6% or 4% by weight of the antiperspirant composition of empty fragrance carriers. In some instances, it may be desirable to incorporate a mixture of empty fragrance carriers and full fragrance carriers in the antiperspirant composition, wherein the empty fragrance carriers may be included to achieve the desired overall particulate concentration without the risk of perfume over-dosing.

In some instances, it may be desirable to provide a mixture of fragrance carriers and native starch powders to achieve the desired particle concentration. For example, from about a 20:80 to 80:20 (fragrance carrier to starch) mixture might be utilized. In some instances, a 50:50 mixture might be utilized and in other instances the native starch powders might have a concentration equal to about or less than 6% by weight of the antiperspirant composition while the concentration of the fragrance carriers might be equal to about or less than 9% by weight of the antiperspirant composition.

A wide variety of perfume components may be used with the fragrance carriers, including but not limited to volatile perfume components having a boiling point at normal pressure of less than about 260° C., more preferably less than about 250° C., and perfume components having significant low odor detection threshold, and mixtures thereof. The boiling points of many perfume components are given in, e.g., “Perfume and Flavor Chemicals (Aroma Chemicals),” Steffen Arctander, published by the author, 1969.

Other Liquid Materials

While it may be desirable for the liquid materials of the antiperspirant composition to consist essentially of or be primarily formed from non-volatile silicone fluids, the liquid activation enhancer and optionally liquid fragrance materials, it is contemplated that other liquid materials may be optionally included in a deodorant or antiperspirant composition. The liquid materials of the antiperspirant composition may comprise less than 30%, 20%, 10%, or less than 5% by weight of liquid materials other than non-volatile, silicone fluids. Said in another way, the liquid materials of the antiperspirant composition may comprise more than 70%, 75%, 80%, 85%, 90% or about 100% by weight of non-volatile silicone fluids.

It is believed that a deodorant or antiperspirant composition whose liquid materials comprise too much of a volatile silicone fluid may lead to an increased propensity for the appearance of a residue due to the evaporation of the volatile silicone fluid. A deodorant or antiperspirant composition may comprise less than 10%, 5%, 1%, or 0.5% by weight of a volatile silicone fluid. A deodorant or antiperspirant composition may be substantially or completely free of a volatile silicone fluid.

A deodorant or antiperspirant composition may optionally comprise one or more silicone gums. A silicone gum may be added to a deodorant or antiperspirant composition to further increase substantivity of the antiperspirant composition and/or increase the drop size of the aerosol spray particles and/or increase deposition on the skin. However, formulating a deodorant or antiperspirant composition with a silicone gum in combination with relatively high concentrations of a non-volatile silicone fluid and/or relatively high concentrations of total particulates may involve a number of tradeoffs. For example, too much of a silicone gum may dramatically increase viscosity of the antiperspirant composition and the risk of clogging of the container actuator and/or valve, particularly where there is already a relatively high concentration of total particulates. Furthermore, too much of a silicone gum may reduce the diameter of the spray making it more difficult for a user to achieve complete coverage of an axillia (typically a 7.5 cm×12.5 cm area) during application as well as potentially creating regions of high antiperspirant composition dosage, thereby negatively impacting skin feel. Still further, the amount of gum required to control the deposition on skin and diameter of the spray pattern is dependent on the level and/or type of propellent, with the amount needed generally increasing as the propellant level and pressure increases.

Given the one or more potential challenges associated with incorporating a silicone gum, a deodorant or antiperspirant composition may be substantially or completely free of silicone gum materials. When inclusion of a silicone gum is desirable for antiperspirant products with less than about 70% propellant, a deodorant or antiperspirant composition may have a concentration from about 0.05% or 0.075% to about 0.75%, 0.5%, 0.4%, 0.3%, or 0.2% of a silicone gum by weight of the antiperspirant composition. When inclusion of a silicone gum is desirable for antiperspirant products with more than about 70% propellant, a deodorant or antiperspirant composition may have a concentration from about 0.3% or 0.5% to about 3.0%, 2.5%, 2%, 1.5%, or 1.2% of a silicone gum by weight of the antiperspirant composition. The silicone gum material may have a viscosity from about 100,000 centistokes to about 10,000,000 centistokes at 25° C.

If a silicone gum is included, any silicone gum having a viscosity within the ranges described herein may be used, provided it is soluble in the liquid carrier, propellant or a combination thereof of the antiperspirant composition. Some suitable silicone gums include silicone polymers of the dimethyl polysiloxane type, which may have other groups attached, such as phenyl, vinyl, cyano, or acrylic, but the methyl groups should be in a major proportion. Silicone polymers having a viscosity below about 100,000 centistokes (molecular weight below about 100,000) at 25° C. are not considered silicone gums here but are rather, typically, considered a silicone fluid. One non-limiting example of silicone gum suitable for use is a silicone/gum fluid blend comprising a dimethiconol gum having a molecular weight form about 200,000 to 4,000,000 along with a silicone fluid carrier with a viscosity from about 0.65 to 100 mm² s⁻¹. An example of this silicone/gum blend is available from Dow Corning, Corp. of Michigan, USA under the trade name DC-1503 Fluid or XIAMETER® PMX-1503 FLUID (85% dimethicone fluid/15% dimethiconol). Other silicone gums materials include SF1236 Dimethicone, SF1276 Dimethicone, and CF1251 Dimethicone available from Momentive Performance Materials, Inc. of NY, USA.

A deodorant or antiperspirant composition is preferably substantially or completely free of water added as separate ingredient (i.e., anhydrous), as too much added water may result in several deleterious effects such as: 1) increasing the propensity for antiperspirant active particulates to agglomerate (thereby increasing the propensity for clogging), and 2) reducing dry feel on skin. It will be appreciated that even an anhydrous antiperspirant composition may still contain some water that is bound with an ingredient (e.g., antiperspirant active, starch, etc.) otherwise added to the antiperspirant composition. Additional examples of antiperspirant or deodorant compositions can be found in US 2015/0023883, incorporated by reference.

### Test Methods

### Total Mass Flow Rate

This measurement method is preferably utilized with aerosol antiperspirant products comprising a continuous actuator, meaning actuating the actuator results in a continuous rather than intermittent spray. At least four aerosol antiperspirant product samples are tested. The product samples are shaken as directed and the actuator is actuated for 2 to 3 seconds, after which each product sample is weighed to measure its mass using any suitable device, such as an analytical balance. The product samples are then immersed in a constant-temperature (25° C.) bath until the internal pressure stabilizes at a temperature of 25° C. The product samples are then removed from the bath and excess moisture is removed by blotting with a paper towel. The products samples are shaken if directed and the actuator is actuated for 5 seconds, which may be accurately timed by use of a stopwatch. Each product sample is again weighed, after which the product samples are returned to the constant-temperature bath. The process of bathing, actuating, and weighing is repeated three times for each product sample. The average total mass flow rate may be calculated from the spray time period (5.0 seconds) and the difference in mass before and after each five second spray, averaged across the four product samples and three repetitions per product sample.

### Mass Flow Rate

This measurement method is preferably utilized with aerosol antiperspirant products comprising a continuous actuator, meaning actuating the actuator results in a continuous rather than intermittent spray. At least four aerosol antiperspirant product samples are tested. The product samples are shaken if directed and then immersed in a constant-temperature (25° C.) bath until the internal pressure stabilizes at a temperature of 25° C. The product samples are then removed from the bath and excess moisture is removed by blotting with a paper towel. Each product sample is weighed to measure its mass using any suitable device, such as an analytical balance. Twelve large plastic bags (one for each product sample times three repetitions) having a suitable volume, such as a 1 L Ziploc brand bag (or a Whirl-Pak Write-on 55 ounce bag, Part #B01195WA available from Nasco, Inc), are weighed to measure their mass using any suitable device, such as an analytical balance. Each product sample is shaken if directed and sprayed into one of the bags for a period of 5 seconds in a manner that minimizes antiperspirant composition from exiting the bag. For example, the opening thru which the spray enters the bag may be limited to about 5 cm. The 5 second spray time period may be accurately measured using a stopwatch. Following the 5 second spray period, the antiperspirant composition is allowed to settle within the bag and the bag remains open for at least 1 minute but not longer than 2 minutes in order to allow the liquid propellant to evaporate. The weight of the bags and their contents are weighed to measure their mass, and the product samples are also weighed. The average mass flow rate of the antiperspirant composition may be determined using the following equation which is averaged across the four product samples and the three repetitions per product sample:

Mass Flow Rate of Antiperspirant Composition (g/sec)=Weight of Bag and Antiperspirant Composition−Weight of Bag/5 seconds

### Antiperspirant Composition Deposition Efficiency, Amount Dispensed, and Amount Deposited

At least four aerosol antiperspirant product samples are tested. The product samples are shaken if directed and the actuator is actuated for 2 to 3 seconds, after which each product sample is weighed to measure its mass using any suitable device, such as an analytical balance. The product samples are then immersed in a constant-temperature (25° C.) bath until the internal pressure stabilizes at a temperature of 25° C. as determined by constancy of internal pressure. At least twelve filter papers, such as Whatman 150 mm (diameter) Filter Paper available under the catalog number 1003-150 from the Whatman Company of the UK, are weighed to measure the mass of the filter using any suitable device, such as an analytical balance. The product samples are removed from the bath, and any excess moisture is removed by blotting with a paper towel. The product samples are shaken if directed, and the product sample is positioned approximately 15 cm away from one of the filter papers, which is preferably weighted and/or fixtured to assure the filter paper does not move during spraying. The actuator of the product sample is actuated for 5 seconds which may be accurately timed using a stopwatch. It will be appreciated, however, that other spray times may be substituted. For example, a two second spray time period might be used to better approximate the amount dispensed/deposited during a typical use cycle by a consumer. The spray from the product sample should be centered on the center of the filter paper. After spraying, the filter paper and product sample are weighed to measure the mass using any suitable device, such as an analytical balance. The steps of bathing, weighing, and actuating are repeated three times for each of the product samples. The average antiperspirant composition efficiency may be calculated using the following equations, averaged across the four product samples and the three repetitions per product sample:

Amount Dispensed (g)=Product Sample Weight Before Spraying−Product Sample Weight After Spraying

Amount Deposited (g)=Filter Paper Weight After Spraying−Filter Paper Weight Before Spraying

$$Antiperspirant Composition Deposition Efficiency ( \% ) = 100 \times \frac{Amount Deposited}{A mount Dispensed * Antiperpsirant Composition Weight \%}$$

To evaluate antiperspirant compositions that have been previously packed in other aerosol containers, the antiperspirant composition may be acquired by the following process. The overcap of the container is removed. The top of the container is punctured using any suitable tool, such as an AC-PD Aerosol Can Puncturing Device available from Aero-Tech Laboratory Equipment Company, LLC of Missouri, USA. The puncture needle is fully extended into the container, and the puncture needle is slowly retracted to permit the gaseous propellant to evacuate the container. Once the puncture needle is completely retracted from the container, the puncturing device can be removed from the container, and the propellant will continue to escape from the puncture in the container. All the propellant is allowed to evacuate from the container before removing 20 grams of the remaining antiperspirant composition for addition to the glass container. It may be necessary to combine antiperspirant composition from multiple containers should there not be 20 grams of material in a single package.

The long term settling height is then easily measured using a clear ruler (although any appropriated measuring device is possible) and is defined as the distance from the top of the antiperspirant composition powder pack to its bottom. Care should be taken during this process to prevent significant agitation that would redisperse the powder pack. The short term settling height is measured by first shaking the glass container vigorously for 30 seconds to achieve complete dispersion of the antiperspirant composition. The glass container is then placed on a flat surface without further agitation for 2 minutes (±5 seconds). The short term settling height is then easily measured at that time using a clear ruler (although any appropriated measuring device is possible) and is defined as the distance from the top of the antiperspirant composition powder pack to its bottom.

### EXAMPLES

The following examples are given solely for the purpose of illustration and are not to be construed as limitations of the invention as many variations thereof are possible without departing from the spirit and the scope of the invention.

Examples 1 to 3 describe some non-limiting examples of antiperspirant compositions comprising a liquid activation enhancer.

TABLE 2
Antiperspirant Compositions (Ex. 1-3)

| Ingredient | EX 1 | EX 2 | EX 3 |
|---|---|---|---|
| Aluminum Chlorohydrate¹ | 26.37% | 26.37% | 26.37% |
| Cyclopentasiloxane | 0% | 0% | 0% |
| Dimethicone² | 43.5% | 38% | 38% |
| Isopropyl Myristate | 9% | 9% | 9% |
| Hydrophilic tapioca material³ | 12% | 12% | 12% |
| Stearalkonium Hectorite⁴ | 4.25% | 4.25% | 4.25% |
| Triethyl Citrate | 1.38% | 1.38% | 1.38% |
| Silicone Gum⁵ | 0.5% | 0.5% | 0.5% |
| Liquid Fragrance Material⁶ | 0% | 5.5% | 5.5% |
| Complexed Beta Cyclodextrin | 3% | 3% | 3% |
| Total | 100 | 100 | 100 |

¹86% assay of anhydrous active, average particle size approximately 15 microns.
²DC 200 Fluid (50 centistoke) available from Dow Corning
³Tapioca Pure from Akzo Nobel
⁴Bentone 38 available from Elementis
⁵DC1503 (a mixture of dimethicone and dimethiconol) available from Dow Corning
⁶Is believed to have contained isopropyl myristate at less than 10% w/w of the liquid fragrance material

Examples 1 and 2 were prepared by mixing a first portion of the cyclopentasiloxane or dimethicone, isopropyl myristate (if present) and disteardimonium hectorite by lightly stirring followed by milling for at least 1 minute using a single head Silverson mill. The triethyl citrate was added next followed by at least five minutes of milling, followed by addition of the aluminum chlorohydrate, a second portion of the dimethicone, the complexed BCDs, tapioca material, dimethicone/dimethiconol and liquid fragrance material. After making the composition, approximately 20 gms thereof was added to a clear glass aerosol bottle (Part #ATL-SC4-48 available from Aero-Tech Laboratory Equipment Co of USA). The glass bottle was sealed with a valve assembly and then approximately 40 gms of isobutane propellant was added to the bottle thru the valve assembly. Each sample was shaken to disperse the composition and hot tanked for four minutes at 130° F. After cooling, the samples were shaken again and allowed to stand for 24 hrs (long term settling) prior to rotational and short term settling testing. Example 3 was prepared by mixing a first portion of the cyclopentasiloxane or dimethicone, isopropyl myristate (if present) and disteardimonium hectorite by lightly stirring followed by milling for at least 1 minute using a single head Silverson Mill. The triethyl citrate was added next followed by at least five minutes of milling, followed by addition of the aluminum chlorohydrate, a second portion of the dimethicone, the complexed BCDs, tapioca material and dimethicone/dimethiconol. Approximately 18.9 gms of this mixture was then added to a clear glass aerosol bottle (Part #ATL-SC4-48 available from Aero-Tech Laboratory Equipment Co of USA) followed by approximately 1.1 gms of the liquid fragrance material. The glass bottle was sealed with a valve assembly and then approximately 40 gms of isobutane propellant was added to the bottle thru the valve assembly. Each sample was shaken to disperse the composition and hot tanked for four minutes at 130 F. After cooling, the samples were shaken again and allowed to stand for 24 hrs (long term settling) prior to rotational and short term settling testing.

Examples 4 to 7 describe some non-limiting examples of antiperspirant compositions comprising a liquid activation enhancer.

TABLE 3
Antiperspirant Compositions (Ex. 4-7)

| Ingredient | EX 4 | EX 6 | EX 6 | EX 7 |
|---|---|---|---|---|
| Aluminum Chlorohydrate¹ | 26.5% | 26.5% | 16.32% | 26.37% |
| Dimethicone² | 38.18% | 38.18% | 32.14% | 43% |
| Isopropyl Palmitate | 9.05% | 0% | 0% | 0% |
| Butyl Stearate | 0% | 9.05% | 0% | 0% |
| Isopropyl Myristate | 0% | 0% | 29.98% | 4% |
| Mineral Oil | 0% | 0% | 0% | 0% |
| Isohexadecane | 0% | 0% | 0% | 0% |
| Octyldodecanol | 0% | 0% | 0% | 0% |
| PPG-14-Butyl Ether | 0% | 0% | 0% | 0% |
| Hydrophilic tapioca material³ | 12.06% | 12.06% | 7.43% | 12% |
| Stearalkonium Hectorite⁴ | 4.27% | 4.27% | 4.25% | 4.25% |
| Triethyl Citrate | 1.39% | 1.39% | 1.38% | 1.38% |
| Silicone Gum⁵ | 0% | 0% | 0% | 0.5% |
| Liquid Fragrance Material⁶ | 5.53% | 5.53% | 5.5% | 5.5% |
| Complexed Beta Cyclodextrin | 3.02% | 3.02% | 3% | 3% |
| Total | 100 | 100 | 100 | 100 |

¹86% assay of anhydrous active, average particle size approximately 15 microns.
²DC 200 Fluid (50 centistoke) available from Dow Corning
³Tapioca Pure from Akzo Nobel
⁴Bentone 38 available from Elementis
⁵DC1503 (a mixture of dimethicone and dimethiconol) available from Dow Corning
⁶Is believed to have contained isopropyl myristate at less than 10% w/w of the liquid fragrance material

Examples 4 to 7 were prepared by mixing a first portion of the dimethicone; one of isopropyl myristate, isopropyl palmitate, butyl stearate, mineral oil, isohexadecane, octyldodecanol and PPG-14-butyl ether; and disteardimonium hectorite by lightly stirring followed by milling for at least 1 minute using a single head Silverson mill. The triethyl citrate was added next followed by at least five minutes of milling, followed by addition of the aluminum chlorohydrate, a second portion of the dimethicone, the complexed BCDs, tapioca material, dimethicone/dimethiconol and liquid fragrance material. After making the composition, approximately 20 gms thereof was added to a clear glass aerosol bottle (Part #ATL-SC4-48 available from Aero-Tech Laboratory Equipment Co of USA). The glass bottle was sealed with a valve assembly and then approximately 40 gms of isobutane propellant was added to the bottle thru the valve assembly. Each sample was shaken to disperse the composition and hot tanked for four minutes at 130° F. After cooling, the samples were shaken again and allowed to stand for 24 hrs (long term settling) prior to rotational and short term settling testing.

Examples 18 and 23 describe some non-limiting examples of antiperspirant compositions comprising C12-15 alkyl benzoate and isopropyl myristate.

TABLE 4
Antiperspirant Compositions (Ex. 8-14)

| Ingredient | EX 8 | EX 9 | EX 10 | EX 11 | EX 12 | EX 13 | EX 14 |
|---|---|---|---|---|---|---|---|
| Aluminum Chlorohydrate¹ | 26.5% | 26.5% | 26.5% | 26.5% | 26.5% | 26.5% | 26.37% |
| 5 Centistoke Dimethicone² | 45.22% | 38.18% | 0% | 0% | 0% | 0% | 0% |
| 10 Centistoke Dimethicone² | 0% | 0% | 38.18% | 0% | 0% | 0% | 0% |
| 20 Centistoke Dimethicone² | 0% | 0% | 0% | 38.18% | 39.57% | 0% | 0% |
| 50 Centistoke Dimethicone² | 0% | 0% | 0% | 0% | 0% | 38.18% | 38% |
| C12-15 Alkyl Benzoate | 2.01% | 9.05% | 9.05% | 9.05% | 9.05% | 9.05% | 0% |
| Isopropyl Myristate | 0% | 0% | 0% | 0% | 0% | 0% | 9% |
| Hydrophilic tapioca material³ | 12.06% | 12.06% | 12.06% | 12.06% | 12.06% | 12.06% | 12% |
| Stearalkonium Hectorite⁴ | 4.27% | 4.27% | 4.27% | 4.27% | 4.27% | 4.27% | 4.25% |
| Triethyl Citrate | 1.39% | 1.39% | 11.39% | 1.39% | 0% | 1.39% | 1.38% |
| Silicone Gum⁵ | -none | none | none | none | none | none | 0.5% |
| Liquid Fragrance Material⁶ | 5.53% | 5.53% | 5.53% | 5.53% | 5.53% | 5.53% | 5.5% |
| Complexed Beta Cyclodextrin | 3.02% | 3.02% | 3.02% | 3.02% | 3.02% | 3.02% | 3% |
| Total | 100 | 100 | 100 | 100 | 100 | 100 | 100 |

¹86% assay of anhydrous active, average particle size approximately 15 microns.
²DC 200 Fluid (5, 10, 20 or 50 centistoke) available from Dow Corning
³Tapioca Pure from Akzo Nobel
⁴Bentone 38 available from Elementis
⁵DC1503 (a mixture of dimethicone and dimethiconol) available from Dow Corning
⁶Is believed to have contained isopropyl myristate at less than 10% w/w of the liquid fragrance material

Examples 8 to 14 were prepared by mixing a first portion of the dimethicone, C12-15 alkyl benzoate, and disteardimonium hectorite by lightly stirring followed by milling for at least 1 minute using a single head Silverson mill. The triethyl citrate was added next followed by at least five minutes of milling, followed by addition of the aluminum chlorohydrate, a second portion of the dimethicone, the complexed BCDs, tapioca material, dimethicone/dimethiconol and liquid fragrance material. After making the composition, approximately 20 gms thereof was added to a clear glass aerosol bottle (Part #ATL-SC4-48 available from Aero-Tech Laboratory Equipment Co of USA). The glass bottle was sealed with a valve assembly and then approximately 40 gms of isobutane propellant was added to the bottle thru the valve assembly. Each sample was shaken to disperse the composition and hot tanked for four minutes at 130° F. After cooling, the samples were shaken again and allowed to stand for 24 hrs (long term settling) prior to rotational and short term settling testing.

### Example: Aerosol Dispenser for Antiperspirant Product with Compressed Air Propellant

An aluminum canister, for example an aluminum canister with an internal volume of 250 mL could be provided along with a bag-on-valve assembly. The bag can be attached to the aerosol valve assembly and then it can be shoved inside the aluminum canister. Next, 50 mL of antiperspirant composition, as described in Examples 1-14, can be added to the bag. Nitrogen propellant or carbon dioxide propellant can be filled into the space between the canister and the bag.

The propellant can be filled to a starting pressure as described in Table 5, below. At ambient temperature, the dispenser can have a spray rate of 0.2 g/sec or less. The mean droplet size can be less than 50 μm at the first spray and the last spray. The propellant can have an ending pressure and spray rate as calculated in Table 5, below. The dispenser can have preferable spray properties if the starting pressure and the ending pressure is greater than or equal to 100 psi. It is assumed that 50% of the pressurized gas will be lost and all of the product will be dispensed.

TABLE 5
Pressure with N2 or CO2 Propellant

| | Ex. A | Ex. B | Ex. C | Ex. D | Ex. E | Ex. F | Ex. G | Ex. H | Ex. I |
|---|---|---|---|---|---|---|---|---|---|
| Starting Pressure (psi) | 100 | 110 | 120 | 130 | 140 | 150 | 160 | 170 | 180 |
| Ending Pressure (psi) | 2.72 | 2.992 | 3.264 | 3.536 | 3.808 | 4.08 | 4.352 | 4.624 | 4.896 |
| N2 spray rate with 50% | 0.0047 | 0.0052 | 0.0056 | 0.0061 | 0.0066 | 0.0070 | 0.0075 | 0.0080 | 0.0084 |
| loss of gas (g/s) | | | | | | | | | |
| CO2 spray rate with 50% | 0.0074 | 0.0081 | 0.0088 | 0.0096 | 0.0103 | 0.0110 | 0.012 | 0.013 | 0.013 |
| loss of gas (g/s) | | | | | | | | | |
| N2 spray rate with 25% | 0.0023 | 0.0026 | 0.0028 | 0.0030 | 0.0033 | 0.0035 | 0.0038 | 0.0040 | 0.0042 |
| loss of gas (g/s) | | | | | | | | | |
| CO2 spray rate with 25% | 0.0037 | 0.0040 | 0.0044 | 0.0048 | 0.0051 | 0.0055 | 0.0060 | 0.0063 | 0.0066 |
| loss of gas (g/s) | | | | | | | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”. All numeric values (e.g., dimensions, flow rates, pressures, concentrations, etc.) recited herein may be modified by the term “about”, even if not expressly so stated with the numeric value.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aerosol antiperspirant or deodorant product comprising:
a dispenser comprising:
a dispenser container at least partially filled with a compressed gas selected from nitrogen, carbon dioxide, and combinations thereof; wherein the dispenser is fitted with a valve assembly comprising: a mounting cup, one or more gaskets, a valve seat, a spring, a housing, and a dip tube;
an ingredient containing reservoir filled with antiperspirant or deodorant composition operatively connected to the dispenser container and the dip tube via a first dip tube and a second tube such that on actuation of the valve assembly the composition and the nitrogen gas travel along the first tube and the second tube respectively, and mix in the valve assembly before exiting the dispenser container via an actuator spray nozzle to an environment or subject; or
the nitrogen gas travels along the second tube into the ingredient containing reservoir carries the composition along the first tube where they mix in the valve assembly before exiting the dispenser container (90) via the actuator spray nozzle (200) to an environment or subject;
wherein the antiperspirant or deodorant composition comprises a non-volatile silicone fluid having a concentration from about 30% to about 70% by weight of the antiperspirant composition, a deodorant or antiperspirant active, an organoclay material and at least one liquid activation enhancer having a Hansen Solubility Parameter for Hydrogen Bonding, δₕ, between about 2 and about 6 and a light transmittance value greater than 90%.

2. The aerosol antiperspirant or deodorant product according to claim 1, wherein the concentration of the liquid activation enhancer is from 2% to 30% by weight of the antiperspirant composition.

3. The aerosol antiperspirant or deodorant product according to claim 1, wherein the liquid activation enhancer has the following formula (I):
R₁—X—R₂
wherein R₁ contains from about 8 to about 20 carbon atoms, X is selected from the group consisting of an alcohol, ester, amide and aryl group, and R₂ is selected from the group consisting of null, H, 1 to 4 carbon atoms, and C₆H₆.

4. The aerosol antiperspirant or deodorant product according to claim 3, wherein the liquid activation enhancer is selected from the group consisting of isopropyl myristate, isopropyl palmitate, ethyl stearate, methyl stearate, propyl stearate, butyl stearate, ethyl myristate, ethyl palmitate, butyl palmitate, propyl stearate, propyl palmitate, methyl stearamide, ethyl stearamide, isopropyl stearamide, ethyl palmitamide propyl palmitamide, stearyl benzoate, benzyl palmitate, benzyl stearate, palmityl benzoate, C12-15 alkyl benzoate, palmityl acetate and combinations thereof.

5. The aerosol antiperspirant or deodorant product according to claim 4, wherein the liquid activation enhancer is selected from the group consisting of isopropyl myristate, isopropyl palmitate, butyl stearate, C12-15 alkyl benzoate and combinations thereof.

6. The aerosol antiperspirant or deodorant product according to claim 1, wherein the organoclay material is selected from the group consisting of modified bentonite, modified hectorite, modified montorlinite and combinations thereof.

7. The aerosol antiperspirant or deodorant product according to claim 1, wherein the antiperspirant composition further comprises a clay activator selected from propylene carbonate, triethyl citrate, methanol, ethanol, acetone, water and combinations thereof.

8. The aerosol antiperspirant or deodorant product according to claim 1, wherein the non-volatile silicone fluid has a concentration from about 30% to about 50% by weight of the antiperspirant composition and has a viscosity at the time of making from about 5×10⁻⁶ m²/s to about 350×10⁻⁶ m²/s.

9. The aerosol antiperspirant or deodorant product according to claim 8, wherein the non-volatile silicone fluid has a viscosity at the time of making from about 5×10⁻⁶ m²/s to about 100×10⁻⁶ m²/s.

10. The aerosol antiperspirant or deodorant product according to claim 1, wherein the non-volatile silicone fluid comprises a polydimethylsiloxane fluid having an average molecular weight from about 500 to about 13,700 at the time of making wherein the polydimethylsiloxane fluid has the following formula (II):
M-D_{X}-M
wherein M is (CH₃)₃SiO, D is ((CH₃)₂SiO) and X is from about 4 to about 183.

11. The aerosol antiperspirant or deodorant product according to claim 1, wherein the antiperspirant composition is substantially free of a volatile silicone fluid.

12. The aerosol antiperspirant or deodorant product according to claim 1, wherein the liquid activation enhancer is C12-15 alkyl benzoate and the non-volatile silicone fluid comprises a polymethylsiloxane fluid having a viscosity less than about 20×10⁻⁶ m²/s at the time of making.

13. The aerosol antiperspirant or deodorant product according to claim 1, wherein the antiperspirant composition is substantially free of mineral oil, isohexadecane, PPG-14 butyl ether and octyldodecanol.

14. The aerosol antiperspirant or deodorant product according to claim 1, wherein the non-volatile silicone fluid comprises a polymethylsiloxane fluid having a concentration from about 30% to about 50% by weight of the antiperspirant composition and wherein the liquid activation enhancer is isopropyl myristate having a concentration from about 2% to about 10% by weight of the antiperspirant composition and wherein the antiperspirant composition further comprises a liquid fragrance material having a concentration from about 4% to about 6% by weight of the antiperspirant composition.

15. The aerosol antiperspirant or deodorant product of claim 1, wherein the dip tube of the valve assembly divides into the first and second tubes seated within a dispenser container, and wherein the first tube, seated within the dispenser, is connected to the ingredient containing reservoir, allowing the composition to be dispensed on actuation of the valve.

16. The aerosol antiperspirant or deodorant product of claim 1, wherein the ingredient containing reservoir is a bag or pouch.

17. The aerosol antiperspirant or deodorant product of claim 1, comprising three or more tubes and at least two ingredient containing reservoirs comprising different ingredients.

18. A method for applying an antiperspirant or deodorant composition comprising:
a. providing the antiperspirant or deodorant product of claim 1;
b. actuating the dispenser thereby dispensing the composition as an ejected composition comprising an average particle size distribution (Dv50) of <50 microns and a mass flow rate of antiperspirant or deodorant composition from about 0.05 g/s to about 0.4 g/s;
c. applying the ejected composition to the underarms.

19. The method according to claim 18, wherein the mass flow rate and the median particle size vary by no more than 30% from the mean value of the first 25% of the canister to the mean value of the last 25% of the canister.

20. The method according to claim 18, wherein the composition is dispensed without substantial clogging in the valve assembly.
